(19) United States
(12) Patent Application Publication
(10) Pub. No.: US 2021/0379397 A1
(43) Pub. Date: Dec. 9, 2021

(54) UV-Mask for Crosslinking Treatment of Keratoconus

(71) Applicant: Robert Epstein, McHenry, IL (US)

(72) Inventor: Robert Epstein, McHenry, IL (US)

(21) Appl. No.: 17/339,991

(22) Filed: Jun. 5, 2021

Related U.S. Application Data
(60) Provisional application No. 63/035,222, filed on Jun. 5, 2020.

(51) Int. Cl. A61N 5/06; A61F 9/00

(52) U.S. Cl. CPC A61N 5/062; A61N 2005/0648; A61N 2005/0661; A61F 9/0017

(57) ABSTRACT

A UV-mask for treating keratoconus. The UV-mask could be made by having an image representing a surface of the patient's cornea with a topography map indicating an area of abnormal corneal thickness. The image is printed onto a sheet. The sheet is cut on or around the area of abnormal corneal thickness to create a transparent window in the sheet. The sheet is further cut on or around the corneal surface to result in a UV-mask. Also disclosed is a UV-mask for use in UV-irradiation treatment of keratoconus. Also disclosed are methods of treating keratoconus in an eye of a patient using a UV-mask.

### TECHNICAL FIELD

This invention relates to the treatment of keratoconus (cornea) by UV irradiation crosslinking.

### BACKGROUND

Keratoconus is a condition of the eye in which the surface of the cornea becomes abnormally shaped. A portion of the cornea becomes thin, whereas another portion of the cornea bulges outward, often in an irregular shape. One of the conventional treatment options is corneal crosslinking to stop the progression of keratoconus. The conventional protocol for corneal crosslinking is called the “Dresden protocol,” which results in crosslinking of collagen fibers in the cornea.

The Dresden treatment is performed by removing the corneal epithelium in a 9 mm diameter disk-shaped region, followed by administering riboflavin eyedrops to the cornea. This riboflavin treatment is continued over 30 minutes so that it soaks into the cornea. After riboflavin soaking, the area is exposed to UV-A light at a fluence of 3 mW/cm² for 30 minutes.

A newer procedure has been developed that allows the riboflavin to soak into the cornea without needing to remove the corneal epithelium. This revised procedure uses riboflavin with vitamin E-TPGS (tocopherol polyethylene glycol succinate) added as a permeation enhancer, which lets riboflavin soak deeper into the cornea. After the riboflavin soaking, the cornea is irradiated with UV-A light to cause the crosslinking bonds to form.

Further advances have been made analyzing the biomechanics of corneal shape change in keratoconus. See Caruso et al, “Topography and Pachymetry Guided, Rapid Epi-on Corneal Cross-Linking for Keratoconus: 7-year Study Results” (2020 January) Cornea 39(1):56-62. Using finite element analysis with application of the elastic plate theory, a three-dimensional model of keratoconus progression was generated. This model was used to design a crosslinking technique using a narrower UV-A beam than that is used in the standard Dresden protocol. With this new protocol, reducing the size of the circular disk-shaped UV-A beam to less than 9 mm performed better at normalizing the corneal curvature.

### SUMMARY

This invention addresses the need for improved treatment of keratoconus using a UV-mask for corneal crosslinking. Keratoconus only affects a portion of the cornea. The affected region is typically irregular in shape and may not even be in the central cornea. This invention seeks to improve corneal crosslinking treatment by focusing on the area of the disease.

There are multiple aspects to the invention as disclosed herein. In one aspect, the invention is a method of making a UV-mask for treating keratoconus in a patient. The method comprises having an image representing a surface of the patient's cornea. The image shows a topography map indicating an area of abnormal corneal thickness. A topography map shows the shape of the cornea. There are a variety of different types of topography maps that could be used. One example is a pachymetry map that indicates corneal thickness by way of different colors (e.g. a color scheme in which cooler colors indicate surface flatness and warmer colors indicate surface steepness). Particularly useful is an axial/sagittal map of the curvature of the front surface of the cornea. A variety of ocular imaging apparatus are available to produce such images, such as the Oculus Pentacam, Zeiss Atlas, NIDEK OPD-Scan, Visionix VX130, Orbscan Topographer, and Topcon Corneal Analyzer.

The image is printed onto a sheet. Printing of the image may be performed by any conventional means, such as inkjet or laser printing. The image may be in any color format, such as full color, black/white, or grayscale. The image or the parameters of the UV-mask design (as described below) could be stored in software so that repeat copies could be made.

Any type of sheet suitable for applying onto the cornea and capable of blocking UV-light could be used. The sheet may be made of any suitable flexible material, such as plastic film, paper, or biologic material (e.g. collagen). Preferably, the sheet is very thin, having a thickness in the range of 0.05-0.2 mm, and preferably, in the range of 0.09-0.15 mm. The sheet has an area of at least 2×2 cm in size. The sheet may have any shape, but square and rectangular sheets are the most common. For paper sheets, various types may be suitable for use. For example, in U.S. industry standards, paper sheets having a weight rating in the range of 18-36 lb., or 20-32 lb., or 22-26 lb. weight could be used. The sheet may be any suitable color to render the image. For example, the sheet may be white color.

The image printed on the sheet must be appropriately sized for applying onto the cornea. Thus, the image is printed such that the lateral width of the corneal surface is in the range of 5-12 mm, or 7-10 mm. The sheet is cut on or around the area of abnormal corneal shape (e.g. abnormal thickness or curvature) to create a window in the sheet (this means the image representation of the area of abnormal shape, not the actual cornea). This is sometimes referred to as negative-space cutting.

The sheet may be cut in any suitable manner. Preferably, the cutting is performed by laser cutting. But other examples of cutting techniques that could be used include scissors, knife blade, stamping, etc. Different from the window cut, another one or more cuts are made in the sheet for defining the UV-mask itself. The sheet is cut on or around the corneal surface to result in a UV-mask having a lateral width of 9-17 mm, or 11-15 mm (this means the image representation of the corneal surface, not the actual cornea).

In addition to these cuts, other cuts (one or more deformation cuts) may be made into the UV-mask to increase flexibility and improve conformal fitting onto the cornea surface. Any suitable type of cut could be used, including lines, kerf cuts, notches, etc. These cuts may be located at the edges of the UV-mask. These additional cuts could be made at any appropriate stage of the process. For example, these cuts could be made before the UV-mask is cut out or afterwards. The UV-mask could also have one or more alignment mark (such grooves, creases, lines, scoring, cut edges, notches, incisions, etc.) that would help the clinician apply the UV-mask in its proper orientation.

In another aspect, this invention is a UV-mask for use in UV-irradiation crosslinking treatment of keratoconus. The UV-mask is made as a thin layer having a thickness in the range of 0.05-0.2 mm, and preferably, in the range of 0.09-0.15 mm. The UV-mask has a lateral width of 9-17 mm, or 11-15 mm. The UV-mask has a transparent window having a lateral width in the range of 5-12 mm, or 7-10 mm. The transparent window is positioned completely within the interior of the UV-mask shape. Elsewhere, the mask is opaque to UV light (blocking transmission of at least 95% of UV-A radiation). The UV-mask in this aspect of the invention may have any of the other additional features described above.

This UV-mask could be made by the technique described herein, or any other suitable manner. For example, instead of subtractive manufacturing by successively cutting away material, additive manufacturing techniques could be used. For example, the UV-mask could be made by deposition of one or more successive layers of the mask material according to the predetermined shape. One such example is 3D printing techniques.

In another aspect, the invention is a method of treating keratoconus in an eye of a patient using a UV-mask of the present invention. The method comprises topically applying a corneal crosslinking agent to a cornea. The crosslinking agent may be applied in any suitable manner. Commonly used protocols are continuously or intermittently (e.g. 3 mins) for 30 minutes duration. There are a variety of different types of corneal crosslinking agents that could be used. Riboflavin is the most commonly used. The riboflavin could be mixed with additives such as dextran, trometamol, EDTA, vitamin E-TPGS (tocopheryl polyethylene-glycol succinate), benzalkonium chloride (BAC), Tris, etc. Although isotonic riboflavin solutions are used most commonly, a hypotonic solution could be used to improve absorption by inducing corneal edema.

The method further comprises applying the UV-mask onto the cornea. A gel such as hydroxypropyl methylcellulose, hydroxyethyl cellulose, glycerin, polyethylene glycol, or carboxymethylcellulose could be applied to the UV-mask to enhance adhesion to the cornea surface. The UV-mask is positioned on the cornea such that the transparent window is over a portion of the cornea affected by keratoconus. In embodiments where the UV-mask has deforming cut(s), the UV-mask may be deformed along these cut(s) to improve conformal fitting to the cornea surface.

The cornea and UV-mask is exposed to UV-light such that UV-light is transmitted through the transparent window, but not the opaque portions of the mask. The UV-light irradiation may be performed in any suitable manner. Most commonly, after the crosslinking agent is applied, UV-A irradiation treatment is applied for about 30 minutes. The UV radiation causes crosslinking of corneal collagen fibers under the transparent window portion. But because the UV radiation does not transmit (or is otherwise impeded) through the opaque portions of the mask, those masked areas of the cornea are not crosslinked.

It is common for the patient to move their eyes during the UV-light irradiation procedure. However, being adhered to the cornea, the UV-mask could move with the eye. Thus, it may be possible to perform the UV-light irradiation without changing the direction of the UV-light irradiation to follow eye movement. The UV-mask safeguards that the same area of the cornea continues to be treated even if there is eye movement.

The procedure could be performed by an epithelium-on (“epi-on”) or epithelium-off (“epi-off”) technique. In the “epi-on” technique, the corneal epithelium is left intact during the procedure. In the “epi-off” surgical technique, the corneal epithelium is removed prior to applying the crosslinking agent. This may be necessary to promote adequate diffusion of the crosslinking agent into the cornea. There are several techniques for removing the corneal epithelium for this purpose. These include debridement with a scalpel or rotating brush, pretreatment with ethanol followed by rubbing of the epithelium with a cellulose sponge, laser scraping, etc.

In another embodiment of this treatment method, the UV-mask is used to aid in removing the corneal epithelium over the keratoconus area to be treated. That is, this invention could be implemented for improving the epithelium-off crosslinking technique. In this embodiment, the UV-mask is applied onto the cornea, prior to treatment with the crosslinking agent. A gel such as hydroxypropyl methylcellulose, hydroxyethyl cellulose, glycerin, polyethylene glycol, or carboxymethylcellulose could be applied to the UV-mask to enhance adhesion to the cornea surface. The UV-mask is positioned on the cornea such that the transparent window is over a portion of the cornea affected by keratoconus. One or more alignment marks (e.g. making lines at the 3, 6, 9, and 12 o'clock positions and orienting the UV-mask along those axes according to pre-made ink markings made on the corneal limbus) may help in positioning or adjusting the UV-mask properly. In embodiments where the UV-mask has deforming cuts, the UV-mask may be deformed along these cuts to improve conformal fitting to the cornea surface.

In this embodiment, the clinician uses the UV-mask to mark an outline of the area of the corneal affected by keratoconus. For example, a conventional skin marker could be used to trace the outline onto the cornea. The corneal epithelium is then removed within the outlined area, using any conventional technique, including the ones described above. The UV-mask is then removed and the crosslinking agent is applied to the cornea. Certain crosslinking agents (such as plain riboflavin) will not penetrate into the cornea except where the epithelium has been removed. Such non-penetrating crosslinking agents could be used, such that UV crosslinking would not occur except in the limited area determined by the window of the UV-mask.

The method further comprises exposing the cornea (unmasked) to the UV-light. Additional crosslinking agent may be applied during the UV-light treatment. The UV-light exposure causes collagen fiber crosslinking in the area of the cornea where the epithelium has been removed. This results in more precise crosslinking of the cornea. Also, because less epithelium is removed (compared to the full 9.0 mm diameter epithelium removal in the standard protocol), the post-procedure healing is more rapid. Also, because the non-crosslinked area does not suffer increased hardness, the amount of curvature normalization in the treated area is greater. Depending on the size of the treated area, the initial epithelial healing phase may be reduced from the usual 7 days to less than 5 days, or less than 3 days.

The treatment methods of this invention could result in more rapid improvement of visual acuity than prior treatment techniques. In some embodiments, the treatment results in visual acuity improvement within 30 days, or 15 days, or 10 days, or 5 days. Visual acuity may be assessed by any conventional technique, including best corrected visual accuracy (BCVA), best spectacle-corrected distance visual acuity (BSCDVA), spectacle prescription parameters, measurement of the corneal curvature (K, keratometry), such as the average of five different measurements of the maximal corneal curvature reading over the entire cornea (Kmax5).

### BRIEF DESCRIPTION OF THE DRAWINGS

FIG. 1A shows a sagittal cross-section view of an eye with a normal cornea. FIG. 1B shows an eye affected by corneal keratoconus

FIG. 2 shows a computer-generated topographic map (axial curvature) image of the eye with corneal keratoconus.

FIGS. 3A-3E show how a UV-mask is made using this map image.

FIGS. 4A-4C show how the UV-mask is used for crosslinking treatment of the cornea.

### DETAILED DESCRIPTION OF AN EXAMPLE EMBODIMENT

To assist in understanding the invention, reference is made to the accompanying drawings to shown by way of illustration specific embodiments in which the invention may be practiced. The drawings herein are not necessarily made to scale or actual proportions. For example, lengths and widths of the components may be adjusted to accommodate the page size.

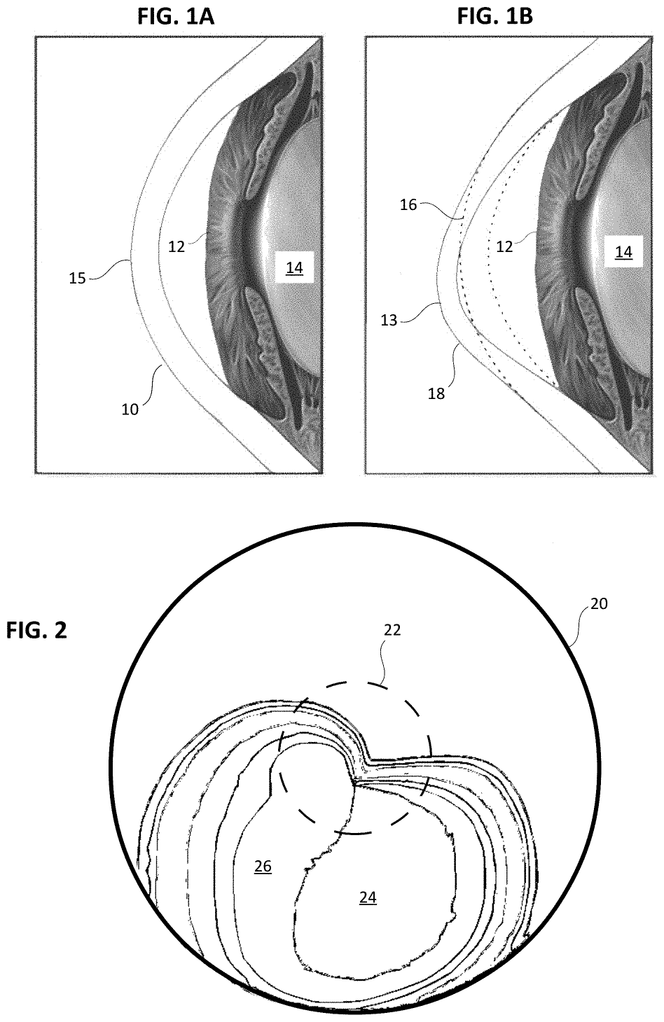

FIG. 1A shows a sagittal cross-section view of an eye with a normal cornea 10. Other parts of the eye seen here are the iris 12 and lens 14. The steepest part of the cornea is the apex 15. FIG. 1B shows an eye affected by corneal keratoconus. As seen here, the cornea 18 is abnormally shaped with an inferior steepening bulge. (For comparison, dotted line 16 shows the normal shape of the cornea.) The steepest part of this abnormally-shaped cornea is the apex 13, which has shifted inferiorly (downward) compared to the apex 15 of the normal cornea 10 of FIG. 1A. This invention seeks to perform cross-linking focused at the keratoconus bulge, while avoiding cross-linking on unaffected regions of the cornea 18.

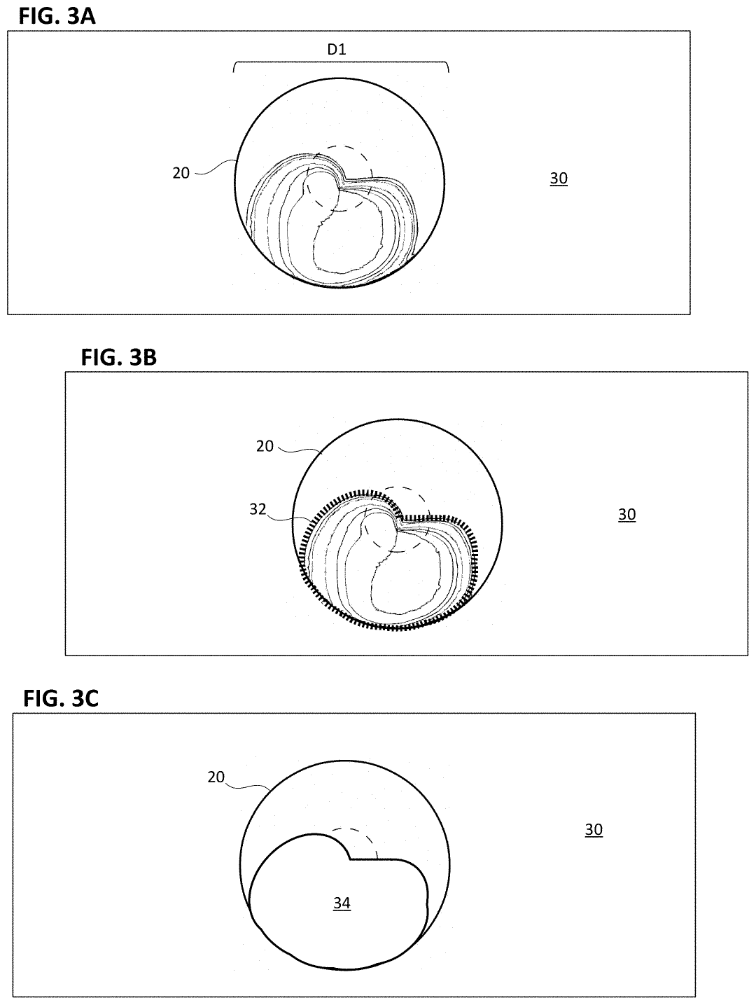

FIG. 2 shows a computer-generated topographic map (axial curvature) image 20 of the eye with corneal keratoconus. The dashed line 22 represents the pupillary zone. This topographic map image 20 shows the front surface of the cornea with contour lines indicating the relative steepness of the corneal surface. Region 24 is the steepest part of the cornea, region 26 is the next steepest part, and further contour lines representing gradual concentric flattening of the cornea in the outward radial direction from region 24. These contour lines represent the area of keratoconus targeted for UV cross-linking treatment with ultraviolet (UV) light.

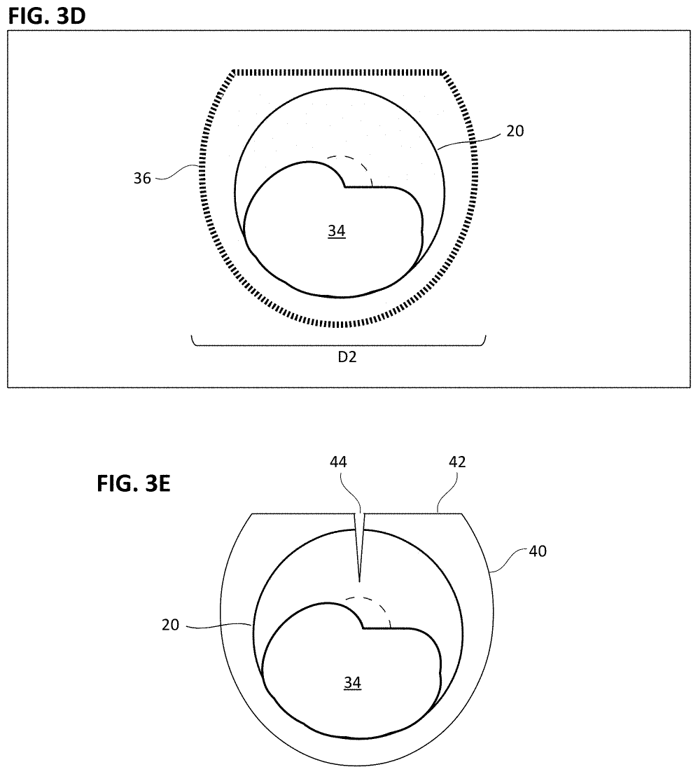

FIGS. 3A-3E show how a UV-mask is made using this map image 20. FIG. 3A shows the map image 20 printed onto a paper sheet 30. As printed, the size of image map 20 is reduced such that the diameter D1 measures about 8.5 mm wide. In FIG. 3B, the sheet 12 is cut along the path shown by dotted line 32 by burns made using a conventional, high precision green argon laser (such as those typically used for retinal laser treatment). As seen in FIG. 3C, when the cutout section is detached from paper sheet 30, this leaves a window 34 in paper sheet 30. This window 34 is in the shape of the area affected with keratoconus and the focus of UV-light crosslinking treatment.

With window 34 cut into the sheet 30 within map image 20, FIG. 3D shows how the UV-mask is cut out from the paper sheet 30. With burns using the green argon laser, the sheet 30 is cut along the path shown by dotted line 36 (i.e. the mask outline). The mask outline is designed such that the diameter D2 measures about 13 mm wide to fully cover the cornea. As shown in FIG. 3E, the cutout portion is detached from the sheet 30 to yield a UV-mask 40. There is also an orientation notch 42 at the top side (superior) of the UV-mask 40 to indicate the proper orientation for applying the UV-mask 40 to the eye. Using scissors, a small, radially extending cut 44 (kerf cut) is made on the superior side to allow bending, stretching, or warping of the UV-mask 40 for better fit onto the curved corneal surface. Optionally, additional kerf cuts could be made on UV-mask 40. Also, orientation markings could be made onto UV-mark 40, such as small lines or spots indicating the vertical or horizontal axes (e.g. at the 3, 6, 9, and 12 clock positions).

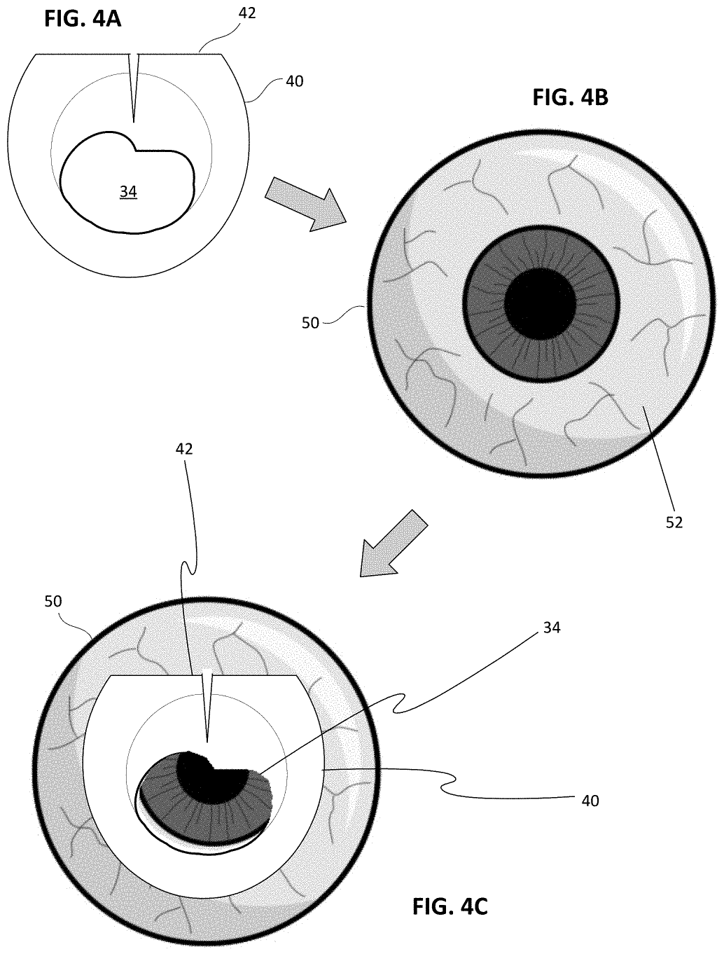

FIGS. 4A-4C show how the UV-mask 40 is used for crosslinking treatment of the cornea. The sclera 52 around the cornea is marked to indicates its vertical axis (0-180°) and horizontal axis (90-270°). The cornea is pretreated with riboflavin/vitamin E-TPGS (tocopheryl polyethylene-glycol succinate) and allowed to soak into the cornea for 20 minutes. Because vitamin E-TPGS functions as a permeation enhancer for riboflavin, removal of corneal epithelium (epi-off technique) is not needed. See Caruso et al, “Transepithelial Corneal Cross-Linking With Vitamin E-Enhanced Riboflavin Solution and Abbreviated, Low-Dose UV-A: 24-Month Clinical Outcomes” (February 2016) Cornea, 35(2):145-150. After the soaking, excess riboflavin-vitamin E is washed off. Alternatively, riboflavin alone could be used instead of riboflavin/vitamin E, but this requires removal of the corneal epithelium to allow penetration of riboflavin into the cornea.

FIG. 4A shows the UV-mask 40 ready for use. The UV-mask 40 is wetted with saline solution. In FIG. 4B, the UV-mask 40 is laid onto to the cornea surface with the notch 42 oriented on the superior side and positioned according to the axis markings made on the sclera 52. The position of UV-mask 40 is adjusted such that window 34 lies over the area of keratoconus targeted for crosslinking treatment. The UV-mask 40 is gently dried with a small wicking sponge. The position of UV-mask 40 should not move on the cornea. Optionally, a small dab of hydroxypropyl methylcellulose gel may be applied to the back of UV-mask 40 to enhance adhesion to the cornea.

Covered with the UV-mask 40, the cornea is then exposed to UV-A radiation for 5 minutes. The UV radiation in combination with riboflavin causes collagen fibers in the cornea to form crosslinks. The UV-mask 40 is then removed to perform another cycle of this treatment procedure. That is, riboflavin soaking, then applying the UV-mask 40, and then UV-A irradiation.

### Clinical Case Examples

Case #1: A 35 year-old woman had keratoconus in both eyes and was suffering vision loss therefrom. Two years earlier, she had been treated with scleral contact lenses with some improvement. But the contact lenses became too uncomfortable for her. She continued to suffer vision loss over 18 months while wearing spectacles. Approximately a year ago, she was afflicted with Guillain-Barre syndrome, from which she had mostly recovered. In her initial eye examination, visual fields were normal.

In her pre-treatment visual acuity assessment, best spectacle-corrected distance visual acuity (BSCDVA) in her left eye (OS) was 20/200 and her resulting eyeglass prescription with best correction (Manif-Rx) was −8.75+2.75×100. Her OS maximal corneal curvature (based on the average of five measurements taken on the same day, Kmax5) was 50.3 diopters (D). A custom-made UV-mask was made and “epi-on” crosslinking was done. On post-treatment day one, her BSCDVA for OS was 20/20, and Manif-Rx −6.00+1.25×95, and Kmax5=50.0 D.

Her right eye (OD) visual acuity was BSCDVA 20/30, Manif-Rx −7.00+0.50×56, and Kmax5=52.46 D. Her right eye was treated with “epi-on” transepithelial crosslinking using a custom-made UV-mask. On post-treatment day one, her vision improved to BSCDVA 20/25+, Manif-Rx −7.50+0.50×50, and Kmax5=52.86 D. On her post-treatment day 13, her vision improved further to BSCDVA 20/20, Manif-Rx −7.25+0.50×50, and Kmax5=50.25 D

Case #2: A 56 year-old man had keratoconus in the left eye with visual acuity of BSCDVA 20/40-, Manif-Rx −5.00+2.50×15, and Kmax5=58.82 D. He was initially treated with standard epithelium-off Dresden protocol by riboflavin crosslinking. But this treatment failed to arrest keratoconus progression. His OS vision worsened to BSCDVA 20/50, Manif-Rx −4.75+5.50×15, and Kmax5=63.82 D.

Using a custom-made UV-mask, he was treated by transepithelial crosslinking with riboflavin/vitamin E-TPGS. On his post-treatment day 60, his OS vision improved to BSCDVA 20/40, Manif-Rx −5.25+3.75×10, and Kmax5=62.06 D. In addition, his right eye was also treated riboflavin/vitamin E-TPGS solution crosslinking with a custom-made UV-mask. Prior to treatment, his OD vision was 20/30-, Manif-Rx −9.00+5.50×15, and Kmax5=80.26 D. On his post-treatment day 62, his OD vision improved to BSCDVA 20/25, Manif-Rx −9.00+4.75×180, and Kmax5=75.18 D.

Case #3: A 51 year-old man had LASIK surgery 18 years ago with good results. His vision had been excellent until 2 years prior. Since then, his visual acuity had declined. He had been prescribed eyeglasses to correct his worsening astigmatism. Upon initial examination, his right eye visual acuity was BSCDVA 20/50, Manif-Rx −5.00+8.00×150, and Kmax5=80.86 D. Using a custom-made UV-mask, his right eye was treated by transepithelial crosslinking with riboflavin/vitamin E-TPGS solution. On his post-treatment day two, his OD visual acuity was improved to BSCDVA 20/40+, Manif-Rx −5.00+8.25×152, and Kmax5=79.10.

Summary: In the standard Dresden protocol, visual acuity is typically unchanged (merely stopping progression) or at best, improvements occur after months or years after the procedure. However, use of the methods described herein results far exceed those achieved by the Dresden protocol using a 9 mm UV-A beam with riboflavin crosslinking. In the above examples, there is rapid halt of disease progression and improvement in visual acuity within days to weeks.

The description and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of making a UV-mask for treating keratoconus in a patient, comprising:
having an image representing a surface of the patient's cornea with a topography map indicating an area of abnormal corneal thickness;
printing the image onto a sheet;
cutting the sheet on or around the area of abnormal corneal thickness to create a transparent window in the sheet;
cutting the sheet on or around the corneal surface to result in a UV-mask.

2. The method of claim 1, wherein the image is printed on the sheet such that the lateral width of the corneal surface is in the range of 5-12 mm.

3. The method of claim 1, wherein the resulting UV-mask has a lateral width of 9-17 mm.

4. The method of claim 1, wherein the topography map is an axial/sagittal map of the curvature of a front surface of the cornea.

5. The method of claim 1, further comprising making a deformation cut in the UV-mask.

6. The method of claim 1, further comprising making an alignment mark on the UV-mask for correctly orienting the UV-mask on the cornea.

7. The method of claim 1, wherein the sheet is paper.

8. The method of claim 7, wherein the paper has a thickness in the range of 0.05-0.2 mm.

9. The method of claim 1, wherein the UV-mask is opaque to UV light except in the transparent window.

10. The method of claim 1, wherein cutting the sheet to create a transparent window is performed by laser cutting.

11. The method of claim 1, wherein cutting the sheet on or around the corneal surface is performed by laser cutting.

12. A method of treating keratoconus in an eye of a patient using the UV-mask of claim 1, using a procedure comprising the steps of:
topically applying a corneal crosslinking agent to a cornea of the eye;
applying the UV-mask onto the cornea;
positioning the UV-mask on the cornea such that the transparent window is over a portion of the cornea affected by keratoconus;
exposing the cornea to the UV-light such that UV-light is transmitted through the transparent window, but not the opaque portion of the mask.

13. The method of claim 12, wherein the step of applying the UV-mask onto the cornea is performed after topically applying the corneal crosslinking agent to the cornea.

14. The method of claim 12, wherein visual acuity in the treated eye improves within 5 days after the procedure.

15. The method of claim 12, wherein the UV-mask has a deformation cut and, the procedure further comprises the step of deforming the UV-mask to fit the cornea.

16. The method of claim 12, wherein the UV-mask has an alignment mark, and the procedure further comprises the step of orienting the UV-mask on the cornea according to the alignment mark.

17. A method of treating keratoconus in an eye of a patient using the UV-mask of claim 1, using a procedure comprising the steps of:
applying the UV-mask onto the cornea;
positioning the UV-mask on the cornea such that the transparent window is over a portion of the cornea affected by keratoconus;
marking on the cornea through the transparent window, an outline of an area affected by keratoconus;
removing the corneal epithelium over the area of keratoconus defined by the marked outline;
exposing the cornea to the UV-light.

18. The method of claim 17, wherein the step of applying the UV-mask onto the cornea is performed before topically applying the corneal crosslinking agent to the cornea.

19. The method of claim 17, wherein visual acuity in the treated eye improves within 5 days after the procedure.

20. The method of claim 17, wherein the UV-mask has a deformation cut and, the procedure further comprises the step of deforming the UV-mask to fit the cornea.
